(19) United States
(12) Patent Application Publication
(10) Pub. No.: US 2010/0221190 A1
(43) Pub. Date: Sep. 2, 2010

(54) METHOD FOR PRODUCING A PARTICLE COMPRISING A GAS CORE AND A SHELL AND PARTICLES THUS OBTAINED

(75) Inventors: Marcel Rene Bohmer, Eindhoven (NL); Suzanna Helena Petronella Maria De Winter, S-Hertogenbosh (NL); William T. Shi, Briarcliff Manor, NY (US); Christopher Hall, Hopewell Junction, NY (US); Nico De Jong, Rotterdam (NL); Johanna Elisabeth theodora Van Wamel, Rotterdam (NL); Marcia Emmer, Rotterdam (NL)

Correspondence Address: PHILIPS INTELLECTUAL PROPERTY & STANDARDS, P.O. BOX 3001, BRIARCLIFF MANOR, NY, 10510, US

(73) Assignee: Koninklijke Philips Electronics N.V., Eindhoven (NL)

(21) Appl. No.: 12/161,703

(22) PCT Filed: Jan. 19, 2007
(86) PCT No.: PCT/IB07/50186

§ 371 Date: Jul. 22, 2008

(30) Foreign Application Priority Data
Jan. 24, 2006 (EP) 06100743.1

(51) Int. Cl. A61K 9/14; A61K 49/22; A61P 43/00

(52) U.S. Cl. 424/9.52; 424/493; 977/840

(57) ABSTRACT

The invention relates to a method for the preparation of particles comprising a gas core and shell which particles are suitable for use as contrast agent and as part of a therapeutic composition, especially for drug delivery. These particles show a surprisingly high activation level on provision of ultrasound energy.

### FIELD OF THE INVENTION

The invention relates to a method for the preparation of particles comprising a gas core and shell which particles are suitable for use as contrast agent and as part of a therapeutic composition, especially for drug delivery.

### BACKGROUND OF THE INVENTION

Ultrasound contrast agents are available for diagnostic purposes. Where the first generations of contrast agents were composed of free air bubbles, the current agents generally consist of a gaseous core and a shell; the shell may be composed of a lipid monolayer, a protein such as human serum albumin, or a biodegradable polymer. Agents with a polymer shell are often termed hard-shelled agents and they behave differently from, for instance, lipid shelled agents. They give an ultrasound contrast by release of gas from their interior, which only takes place above a certain threshold of ultrasound exposure (e.g. mechanical index and/or pulse length) value. Other agents do not show such a threshold value before they become acoustically active. Having such a threshold value is thought to be beneficial for certain imaging techniques where first an image without contrast agent activity is made. It is also highly desirably for drug delivery purposes, where every observed acoustic burst corresponds to a single drug delivery event. This enables quantification of the amount of drug delivered.

It is often observed that in a sample of polymer shelled bubbles not all gas-filled polymer capsules become acoustically active upon ultrasound irradiation. For references see Bloch et al. Applied Physics Letters, 84, 631, 2004, FIG. 1. Bouakaz et al. (Bouakaz, Versluis, De Jong, Ultrasound in Medicine and Biology, 31, 391, 2005) were unable to detect the activation of especially small (2 μm) capsules at diagnostic frequencies and pressures.

Incomplete gas release from a contrast agent may lead to loss in imaging sensitivity and therapeutic efficacy. A consequence may be unnecessary high dosing. For drug delivery incomplete release may mean that only a fraction of the drug will be released with a high probability that excess drug will accumulate in the liver or spleen rather than at the region of interest.

U.S. Pat. No. 6,896,659 relates to a method of delivering a therapeutic agent to a localized region within a subject using ultrasound to trigger the release of the agent from hollow microbubbles having a specified set of mechanical properties. The agents disclosed in U.S. Pat. No. 6,896,659 have a controlled fragility which is characterized by a uniform wall thickness to diameter ratio that defines a discrete threshold power intensity. U.S. Pat. No. 6,896,659 specifically discloses a method for preparing the microbubbles wherein cyclooctane is used as a solvent in the creation of the microbubbles. This cyclooctane is in a later step removed by lyophilization. Bouakaz et al. Ultrasound in Medicine and Biology, 31, 391, 2005, have found that such bubbles are actually difficult to break up with ultrasonic power. They find that large capsules break at a lower acoustic pressure than small capsules and conclude that a size effect on the fragility of these bubbles is still present. Under normal diagnostic conditions, at least part of the particles that are made by this method do not break up.

WO-A-98/48783 discloses microparticles that may be used as ultrasonic contrast agent and for delivery of drugs into the blood stream. The microparticles have a shell comprising an inner and an outer layer. The particles are prepared in a process comprising the steps of forming a first aqueous dispersion of a biologically compatible material and mixing with a second solution of a biodegradable polymer wherein the second solution comprises a relatively volatile water-immiscible solvent and a relatively non-volatile water-immiscible non-solvent for said polymer. The relatively non-volatile water-immiscible non-solventis typically a C6-C20 hydrocarbon. In the examples cyclooctane is used. As with the particles described in U.S. Pat. No. 6,896,659, these particles are likely to only partly break up under common ultrasonic conditions.

It is desirable to synthesize an agent that can be acoustically activated at a pressure and frequency acceptable in diagnostic imaging and this activation is preferably complete, release most preferred takes place from all the insonated capsules. It is another object of the invention to make the percentage of activation and thus agent release independent of the particle size of the capsules.

### SUMMARY OF THE INVENTION

We have surprisingly found that the stability of shelled particles comprising a gaseous core, is at least party determined by shell thickness and lack of penetration of water through the shell in the core. We have found that it is highly desired to use a specific combination of solvents in the method for preparing these particles.

Therefore the invention in a first aspect relates to a method for the production of particles comprising a gas core and a shell which method comprises the steps of:
a) providing a mixture comprising a shell composition, a first solvent (1) and a second non-solvent (2);
b) combining the mixture of step (a) with an aqueous composition thereby forming an emulsion of the mixture of step (a) in an aqueous phase;
c) applying conditions for volatizing solvent (1)
d) applying conditions for removal of water
e) applying conditions for removing of non-solvent (2),

wherein non-solvent (2) is selected from the group comprising organic compositions that have a vapor pressure significantly lower than water under the conditions of step (d).

In a further aspect the invention relates to particles obtained by this method, their inclusion in contrast agents and therapeutic agents and to contrast agents or therapeutic compositions wherein the majority of particles can be activated by ultrasonic power that has an intensity in a range that is usual for ultrasound diagnostic imaging.

### DETAILED DESCRIPTION OF THE INVENTION

In the context of the invention the following definitions are used.

The present invention will be described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes. Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

### DESCRIPTION OF THE FIGURES

FIG. 1: schematic overview of the set-up used for event counts and echo intensity measurements.

FIG. 2: event count for pla-pfo capsules, prepared and measured as described in example 1.

FIG. 3, average echo intensity of capsules prepared and measured as described in example 1.

FIG. 4: Event count for pla-pfo mixed with pla-peo measured as described in example 2.

FIG. 5: tumor size as determined for example 5.

### DETAILED DESCRIPTION OF THE PRESENT INVENTION

The invention relates to a method for producing particles that are suitable for use as contrast agents or as drug delivery vehicles in pharmaceutical compositions.

In the method according to the invention step (a) comprises providing a mixture comprising a shell composition, a first solvent (1) and a second non-solvent (2).

This mixture is preferably made around room temperature, more preferred at a temperature between from 4 to 30° C.

In the context of the invention solvent (1) is preferably a good solvent for the shell composition. It is preferred that solvent 1 is a good solvent for the polymer forming the shell and non-solvent 2 is a bad solvent for the polymer forming the shell. Solvent (1) preferably dissolves in water to at least some extent. Solvent (1) is preferably a relatively volatile composition.

Solvent (1) is preferably a solvent having a vapor pressure higher than water under the conditions of step (c), more preferably selected from the group comprising dichloromethane, dichloroethane, isopropylacetate, or a combination thereof.

It is believed that non-solvent (2) is present to make particles comprising a gaseous core and a shell (capsules) instead of solid particles. Therefore suitable compositions for solvent (2) are desirably relatively non-volatile compositions wherein the chosen shell composition does not dissolve or only to a very low extent. Contrary to solvent 1 for non-solvent (2) it is highly preferred that the solubility in water is very low to zero.

Non-solvent (2) is selected from the group comprising organic compositions that have a vapor pressure significantly lower than water under the conditions of step (d). More preferred the vapor pressure of non-solvent (2) is at least 5 times lower than that of water under the conditions of step (d). It will be appreciated that non-solvent (2) is selected such that its vapor pressure is still sufficiently high to enable removal under freeze-drying conditions optionally in combination with a suitable reduced-pressure that is preferably easily reached using well-known standard equipment.

This low vapor pressure will ensure that solvent (2) really stays inside the capsule being formed, leading in the end to form a capsule with a hollow gaseous space. Preferably the capsule comprises at least one hollow space. Most preferred the capsule comprises one main hollow space and optionally small other hollow spaces. If non-solvent (2) is disappearing from the capsule before the removal of solvent 1 is complete, the capsules will show too much shrinking, thereby increasing their wall thickness, in step (c).

In a preferred embodiment non-solvent (2) is selected from the group comprising hydrocarbons comprising a carbon chain length of from 10 to 20 carbon atoms. It was found that it is advantageous to select the non-solvent (2) from cyclodecane, decane or a combination thereof. In a most preferred embodiment, non-solvent (2) comprises cyclodecane, even more preferred the non-solvent (2) essentially consists of cyclodecane. In the context of the invention essentially consists of means that at least 80 wt %, preferably 90 to 100 wt % of the non-solvent (2) is cyclodecane.

Optionally in step (a) pre-mixtures are used of solvent (1) and (2) and of the shell composition and solvent (1).

A further step (b) comprises combining the mixture of step (a) with an aqueous composition thereby forming an emulsion of the mixture of step (a) in an aqueous phase.

Preferably the shell composition containing mixture of step (a) is added to an aqueous composition. To create an emulsion, preferably stirring or another form of agitation/shear forces is applied.

Optionally further emulsification treatment is included to form an emulsion with the desired, preferably monodispersed, particle size distribution.

Suitable equipment to obtain such emulsification treatment is for example selected from colloid mills, homogenizers, sonicaters.

Optionally the emulsion either before or after such treatments, is pressed through a glass filter. When desired such treatment may be repeated multiple times.

An alternative embodiment to create the desired particle size with a narrow distribution is using methods that produce monodisperse emulsions such as inkjet technology and emulsification using microchannels or micropores. For microporous membranes a cross-flow might be applied to improve the size distribution. A method to create particles using ink jet technology is for example disclosed in co-pending application IB2005/052098.

In a next step (c) conditions are applied to remove solvent (1). In the context of this application this is also referred to as volatizing solvent (1). Any suitable technique may be applied to remove solvent (1).

It is highly preferred that the conditions are controlled such that water and, especially, non-solvent (2) are not yet removed. The conditions in step (c) are preferably such that the majority of non-solvent (2) is not yet removed, more preferred essentially no non-solvent (2) is removed. Hence it is preferred that in this step no measures are taken to reduce the pressure around the mixture such as by applying a vacuum.

A suitable way to remove solvent (1) is to increase the temperature for example to a temperature from 25 to 35° C., or simply by stirring the mixture for a given amount of time.

Without wishing to be bound by any theory it is believed that whilst the solvent (1) vaporizes the concentration of the shell composition in the emulsion internal phase increases to over the solubility threshold and at such moment in time the shell composition will start to precipitate.

This precipitation then leads to the formation of a shell of polymer at the surface of the emulsion inner phase (emulsion droplet). It is believed that once the majority or all of solvent (1) has vaporized, a shell composition results which covers a core comprising non-solvent (2), water and optionally other ingredients that may have been added at an earlier stage of the process.

In a further step (d) conditions are applied to remove water from the core. This is immediately followed by the removal of non-solvent (2) in step (e).

It is highly preferred that the removal of water and non-solvent (2) are separated in two different steps. In practice it may be unavoidable to have some overlap between these steps but overlap should preferably be avoided. Generally removal of water is obtained e.g. by freeze-drying techniques. Removal of non-solvent (2) may require further reduction of pressure.

After step (e) a composition comprising dried particles results. Following re-suspension a suitable dosage of an agent such as a contrast agent or therapeutic agent comprising these particles, is administered.

Optionally in step (a) or (b) a stabilizing composition is included. Such stabilizing composition is preferably selected from the group of surfactants and polymers comprising for example polyvinyl alcohol, or a combination of at least two surfactants and/or polymers. If such stabilizing agent is included in the process, the process preferably includes a washing step after removal of solvent (1) to remove the stabilizer.

For the shell material biodegradable polymers and combinations thereof are highly preferred. If combinations of more than one polymer are used, one of the polymers preferably has at least one hydrophobic group such as an aliphatic block or side group(s) or, more preferred fluorinated groups. Without wishing to be bound by any theory it is believed that in the preparation process these groups will orient towards the core side of the capsule, providing a hydrophobic interior. This will keep water out of the capsule. The other part of the polymer provides enough mechanical stability for the capsules to allow for the synthesis procedure including re-dispersion and give sufficient stability in vivo. A biodegradable polymer such as poly-lactic acid is well suited for this, other biodegradable polymers include poly-glycolic acid, polycaprolacton and copolymers thereof.

In a preferred embodiment, the polymer composition is a polymer modified with at least one hydrophobic group that is preferably selected from the group comprising fluoride, alkyl chain comprising from 6 to 24 carbon atoms, or a combination of these.

The most preferred polymer is selected from the group comprising polylactic acid with a perfluorinated moiety, polylactic-polyglycolacid copolymers, polycaprolactone, or a combination thereof.

Low molecular weight polymers generally have less entanglements in the shell and will therefore easier to lead to shell rupture upon the application of ultrasound. Provided that the mechanical stability is sufficient molecular weights of less than 10,000 are preferred. Most preferred the molecular weight is from 2,000 to 10,000.

Optionally the polymer comprising the hydrophobic group may be mixed with other polymer to establish desired properties such as a pegylated polymer or options for targeting such as using a biotinylated polymer to allow for targeting. Post-modification to decorate the capsule with ligands is attractive because it ensures that the targeting moieties are located at the outer surface of the capsule.

In a preferred embodiment the particles are provided with a targeting moiety such as an antibody or antibody fragment to enable targeting to a specific site in the human or animal body.

Therapeutic compositions are optionally incorporated in the core, in the shell or on the shell. Most preferred hydrophobic therapeutic compositions are included in the core. Hexadecane or paraffin oils may be used to solubilize a therapeutic composition in the core. Potential drugs that may be included in the particle core include anti-cancer drugs such as deoxyrubicin and paclitaxel which are quite hydrophobic. We have surprisingly found that hexadecane is a very suitable carrier liquid for hydrophobic therapeutical compositions or hydrophobic contrast agents. We have found that such compositions easily stay dissolved or finely dispersed in hexadecane and these compositions will therefore incorporate inside the core of the capsules in a remaining oil phase. Therefore the dissolved composition is released from the particles only after activation with ultrasound. Therefore in a preferred embodiment, the invention relates to the claimed particles further comprising at least one carrier liquid for a therapeutical composition and/or a contrast agent. The most preferred carrier liquid is hexadecane. In another aspect the invention relates to a method according to the invention wherein before step (c), the composition is supplemented with a composition comprising a therapeutic agent and/or a contrast agent, which agents are dissolved in at least one carrier liquid, preferably comprising hexadecane.

In a further aspect the method according to the invention comprises the inclusion of a therapeutical composition, preferably in step (a) or (b).

Even more preferred the therapeutical composition is added in combination with an oil phase, preferably hexadecane or paraffin.

The core of the particles may comprise any gas. Preferably the gas is a biocompatible gas such as air or nitrogen. Alternatively a gas of low solubility may be used, e.g. perfluorocarbon. If a gas of higher solubility is desired, the inclusion of carbon dioxide may be suitable.

To ensure that the particles are easily broken by ultrasonic power the particles preferably have a shell with an average thickness of from 1 to 50 nm for an average radius of from 1 to 5 micrometer. Most preferred the thinnest shell thickness is at most 3% of the average diameter of the particle.

In a further aspect the invention relates to a particle comprising a gas core and a shell, which is obtained by the process according to the invention and as described in more detail above.

In a further aspect the invention relates to an ultrasound contrast agent comprising at least a particle according to the invention. Generally such contrast agent will comprise a multitude of such particles. It is highly preferred that the majority of the particles, even more preferred from 80 to 100% of the particles are particles obtained by the method described above.

In a preferred aspect the invention relates to a particle composition comprising a gas core and a polymeric shell wherein the particle has a diameter of from 0.1 to 5 micrometer, and a shell thickness of from 1 to 80 nm. Such particles can be acoustically activated by application of ultrasound at a mechanical index of at most 3, more preferred at most 1.6, more preferred at most 1.2, even more preferred at most 1.0, even more preferred at most 0.8.

It is preferred that the activation sets off at a mechanical index above 0.2, more preferred between 0.2 and 0.8, even more preferred at a lower limit of between 0.2 and 0.6.

For most ultrasound imaging and ultrasound mediated drug release applications it is desired that the (contrast) agent comprises a particle composition comprising a particle comprising a gas core and a polymeric shell wherein the particle has a diameter of from 0.1 to 5 micrometer, and an average shell thickness which is at most 5%, more preferred at most 4%, of the particle diameter, preferably a shell thickness of from 1 to 80 nm, which particle can be acoustically activated by application of ultrasound above a threshold range, wherein the threshold range starts at a mechanical index of 0.2 such that the particle shows pronounced release of gas below a mechanical index of 1.2.

Preferably the ultrasound contrast agent comprises polymer-shelled particles according to the invention, wherein at least 80%, preferably 80 to 100% of the particles is acoustically activated upon application of ultrasound at a mechanical index of at most 0.8.

In another aspect the invention relates to a therapeutic composition comprising at least one particle according to the invention. Preferably these particles comprise at least one drug composition. Most preferred the therapeutic composition comprises particles as described above wherein at least 80%, preferably 80 to 100% of the particles is acoustically activated upon application of ultrasound at a mechanical index of at most 3, more preferred at most 1.6, more preferred at most 1.2, even more preferred at most 1.0, even more preferred at most 0.8.

Generally this implies that at least 80% of the particles on application of ultrasound releases the gas and optionally further ingredients from the core. It is highly desired that this release is taking place within a short time frame and within a small mechanical index range.

This acoustic activation can be monitored by the event count set up that is described in the examples. In this set up an activation event is qualified and counted when the amplitude of a received scattered signal (from an activated microparticle) is more than twice the noise level of the detection system.

In an exemplary embodiment, the invention relates to a contrast agent or therapeutic composition comprising particles comprising a gas core and polymeric shell, wherein at least 80% of the particles are activated by ultrasound energy, in a mechanical index window of 0.5 units, preferably a window of 0.4 units, more preferred 0.3 units within the mechanical index range of 0.01 to 3, more preferred 0.1 to 2, more preferred 0.4 to 1.6.

Preferably this activation is evidenced by an increase in the event count to at least 50 under the conditions specified in the examples.

This increase in event count preferably corresponds to an increase in echo intensity to at least 1000 times the initial value within the mechanical index window and range as described above.

Optical observations may be made to view the activation of particles and the gas release from their core. The optical set up described by Bouakaz et al and in the current examples may be used. The pronounced release of gas when particles are activated, for example at a MI of about 0.9, is clearly visible in the formation of bubbles. Hence evidence of full activation of all particles may be obtained by first applying ultrasound at a MI of from 0 to 1.2. Following the activation, a further series of pulses is then given at a higher MI of e.g. around 1.6. This second series of pulses does not give rise to visible gas formation if all particles have already been activated previously.

The particles that result after step (e) are usually re-suspended in a suitable liquid before use. If the agent is to be used as a contrast agent or therapeutic agent for animals or humans, it is preferred that the particles are re-suspended in an aqueous physiological salt solution.

A standard ultrasound transducer may be used to supply ultrasound energy. This sound energy may be pulsed but for maximal triggering of drug release it is preferred that the ultrasound energy is provided in a continuous wave. The gas containing particles can be imaged using several pulses of sound under clinically accepted diagnostic power levels for patient safety.

The invention is now illustrated by the following non-limiting examples.

### EXAMPLES

### Acoustic Activity

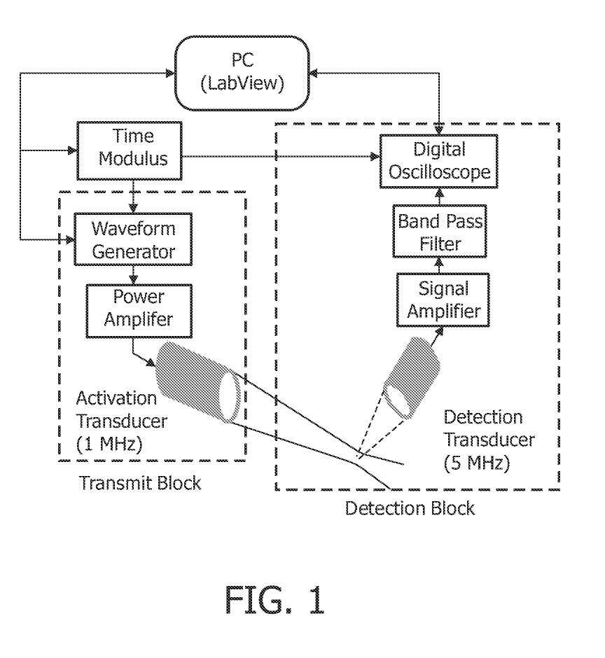

As shown in FIG. 1, the set-up for acoustical measurements consists of three parts: transmit, receive and time modulator blocks. All three blocks are controlled by a personal computer via LabView® (Texas Instrument). A focused sound field is established using a 1.0 MHz cavitation transducer (Panametrices V392) used at a pulse length of 32 cycles. The behavior of activated microcapsules is examined using a passive acoustic detector. The passive detector is composed of a broadband focused transducer (3.8 cm in diameter and 5.1 cm in focal length) with a center frequency of 5 MHz (Panametrics V307) and a broadband low-noise signal amplifier (20 dB). A high-pass filter of 3.0 MHz (TTE HB5-3M-65B) and a low-pass filter of 10.7 MHz (MiniCircuits BLP-10.7) are employed to remove directly transmitted, diffraction-induced 1.0 MHz acoustic signals from the cavitation transducer. A digital oscilloscope (Model LT374L, LeCroy, Chestnut Ridge, N.Y.) is used to digitize the amplified scattering signals with a sampling frequency of 20 MHz.

A time modulator (Four Channel Digital Delay/Pulse Generator; Stanford Research Systems DG535) is used to synchronize the acoustic detector with the activation ultrasound pulses at PRF (pulse repetition frequency) of 2.0 Hz. The activation transducer is mounted horizontally on the sidewall of a rectangular test chamber (20.2×20.2×9.6 cm³) while the acoustic detector is placed vertically and aligned confocally at a right angle with the cavitation transducer. Because both transmit and receive transducers are focused transducers, the detector is very sensitive only to microcapsules in the small confocal region of the two transducers. With the passive technique, the activation threshold and post-activation oscillation (or activation-induced destruction) of microcapsules can be studied by characterizing the waveforms of received acoustic signals, and by analyzing harmonic and noise generation via the spectra of the signals. Activation event counts (or relative activation rates) of microcapsules for every 100 insonations of 1.0 MHz tonebursts were measured by automatically counting received scattered signals using LabView.

Digitized scattered signals from the LeCroy digital oscilloscope are transferred to a PC for further processing. The detection sensitivity (signal to noise ratio) of the experimental system is further increased using a 10th order digital Butterworth band-pass filter with a path band from 2.5 to 6.5 MHz. Therefore, the first harmonic (at the transmit frequency of 1.0 MHz) and the second harmonic are completely removed in the scattered signals. Each filtered signal (containing 3rd, 4th, 5th and 6th harmonics) with a length of 50 micro-second (i.e., 1000 data pints) is summed and averaged for the further enhancement in detection sensitivity (signal to noise ratio). An activation event is qualified and counted when the amplitude of a received scattered signal (from an activated microparticle) is more than twice the noise level of the detection system (i.e., 0.0015 mV or 1.5 micro-volt).

Each sample vial is reconstituted and diluted with a certain amount of de-ionized water, depending on the total number of particles inside the vial. Then a pre-determined small amount of the re-suspended microcapsule sample is injected into a rectangular test chamber using a precision pipette (Eppendorf 200). An amount of 4 liter de-ionized water is used as the carrying and propagation medium in the rectangular test chamber and kept in circulation with a magnetic stirrer at room temperature.

In this way, the number of particles in the test chamber is determined.

In vivo Imaging

The agent was reconstituted in 4 ml of phosphate buffered saline and injected into the tail vein of a rat. 0.2 ml was given for a time of 30 seconds. The agent was imaged at a mechanical index of 0.15 in harmonic mode using a 15 MHz transducer with a bandwidth of 7 MHz. Clear contrast enhancement of the left ventricle was observed and perfusion of the myocardium was detected. The agent circulated for at least 5 minutes.

### Example 1

### Polymer Synthesis

A poly-(lactic acid) (pla) with a perfluorinated moiety at the end was synthesized using 1H,1H-perfluorooctan-1-ol as initiator according to procedures given in reference U.S. Pat. No. 6,329,470 assigned to the State University of New York. We will use the abbreviation pla-pfo for this polymer. A molecular weight of about 6000 was obtained by gel permeation chromatography using known polystyrene size standards for comparison.

pla-pfo Capsules

Pla-pfo was dissolved in dichloromethane to obtain a 5% (w/w) solution (solution A). Cyclodecane was mixed with dichloromethane to obtain a 10% (w/w) mixture (solution B). A quantity of 0.25 g of solution A was mixed with 1 g of solution B. (step a) This mixture was added to 10 g of 0.3% pva solution and emulsified by pressing the mixture through a glass filter. This was repeated 10 times (step b) after which the emulsion was stirred for one hour to evaporate the dichloromethane and complete capsule formation (step c). The emulsion was washed 4 times to remove the excess pva. Centrifugation was used to separate the capsules from the liquid. In all washing steps the capsules were forming a foam layer indicating a lower density for the capsules than for water. 3% polyethylene glycol was added and the samples were lyophilized at a pressure of 1 mbar (step d) and subsequently a pressure of 0.03 mbar (step e) to remove the cyclodecane and re-dispersed before use.

The shell thickness is, based on the initial concentrations, estimated to be 5% of the radius, which is 50 nm for a capsule with a 2 μm diameter.

### Acoustic Measurements

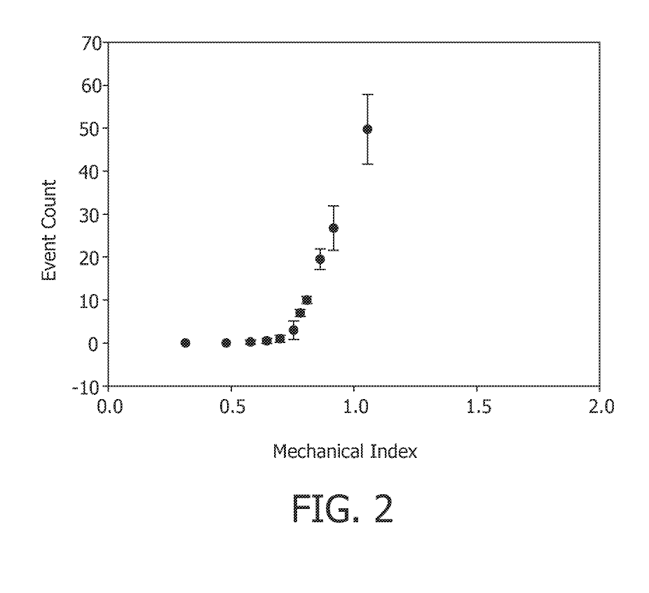

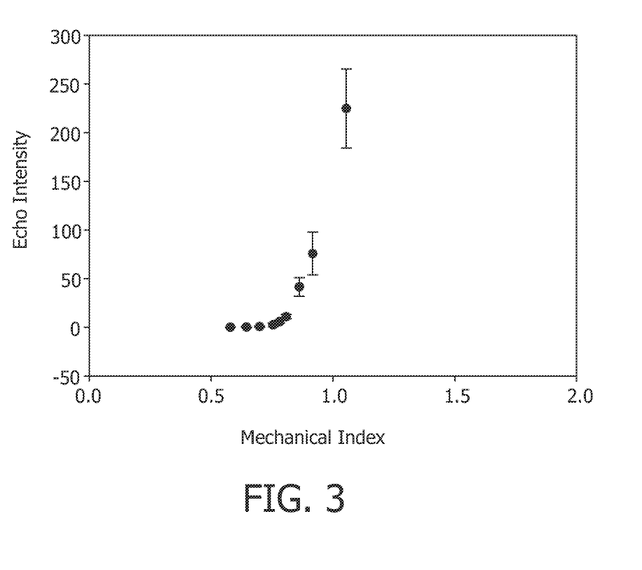

A sample of the agent containing about 20,000 capsules, as determined using a coulter counter, measuring quantitatively between 1 and 30 micrometer, was diluted in 4 liters of water. The number of acoustic events was counted with the set-up described above and in FIG. 1, and plotted as a function of the mechanical index in FIG. 2. A clear threshold value is observed before acoustic events are taking place. This threshold corresponds to a mechanical index of about 0.7 and before a mechanical index of 1.1 the number of events has risen to 50. A steep increase of the acoustic intensity was observed as well, see FIG. 3.

Optical observations were performed using the set-up described by Bouakaz et al. (UMB2005). The thin walled sample was re-dispersed and injected into a 200 micrometer fiber. The fiber was positioned in the focus of a single element transducer operating at 2.25 MHz. High speed camera observations were made for a series of mechanical indices from 0 to 1.4, using 10 cycles at each setting. About 10 particles were in the field of view. At an MI of 0.9 all particles showed pronounced release of gas, in the subsequent ultrasound burst, at an MI of 1.2 no activity was detected showing that all particles had lost the encapsulated gas completely.

### Example 2

Capsules with pla-pfo and pla-peo

Pla-pfo was dissolved in dichloromethane to obtain a 5% (w/w) solution (solution A). pla-peo was dissolved in dichloromethane to obtain a 5% solution (solution B). Cyclodecane was mixed with dichloromethane to obtain a 10% (w/w) mixture (solution B). A quantity of 0.25 g of solution A and 0.25 g of solution B was mixed with 1 g of solution B (step a). This mixture was added to 10 g of 0.3% pva solution and emulsified by pressing the mixture through a glass filter. This was repeated 10 times (step b) after which the emulsion was stirred for one hour to evaporate the dichloromethane and complete capsule formation (step c). The emulsion was washed 4 times to remove the excess pva. Centrifugation was used to separate the capsules from the liquid. In all washing steps the capsules were forming a foam layer indicating a lower density for the capsules than for water. 3% polyethylene glycol was added and the samples were lyophilized at a pressure of 1 mbar (step d) and subsequently a pressure of 0.03 mbar (step e) to remove the cyclodecane and redispersed before use.

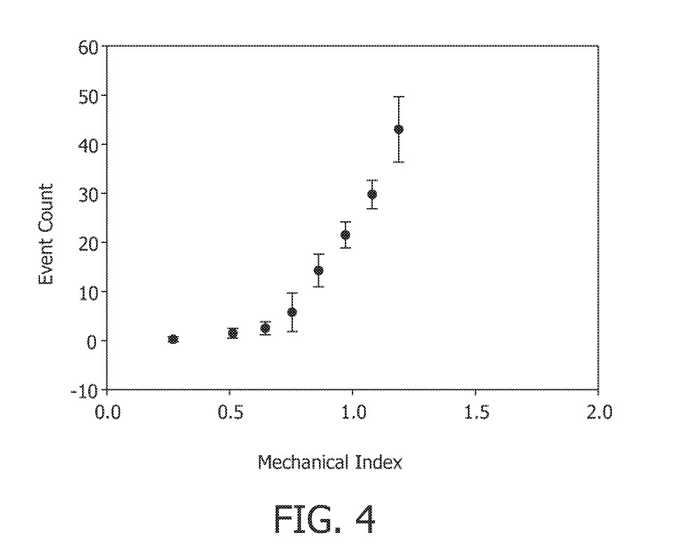

A sample of the agent containing about 20,000 capsules, as determined using a coulter counter, measuring quantitatively between 1 and 30 μm, was diluted in 4 liters of water. The number of acoustic events was counted with the set-up described and plotted as a function of the mechanical index in FIG. 4. A clear threshold value is observed before acoustic events are taking place. This threshold corresponds to a mechanical index of about 0.7 and before a mechanical index of 1.2 the number of events has risen to 45.

### Example 3

### Targeted Contrast Agents

A biotinylated agent was prepared from a mixture of polylactides:polylactide with a fluorinated end group (pla-pfo), pegylated polylactide (pla-peo) and biotinylated, pegylated polylactide (pla-peo-biotin) where the biotine was covalently bound to the pegylated group, the average molecular weight of all polylactides was below 7,000. The polymers were used in a 5:4:1 ratio of pla-pfo:pla-peo:pla-peo-biotin. 0.25 g of a 5% solution of the polymer in dichloromethane was mixed with 1 g 10% cyclodecane in dichloromethane. This mixture was added to 10 g of 0.3% pva solution and emulsified by pressing the mixture through a 1 μm glass filter. This was repeated 10 times after which the emulsion was stirred for one hour to evaporate the dichloromethane and make cyclodecane filled capsules. The emulsion was washed 4 times to remove the excess pva. Centrifugation was used to separate the capsules from the liquid. In all washing steps the capsules were forming a foam layer indicating a lower density for the capsules than for water. 3% polyethylene glycol was added and the samples were lyophilized to remove the cyclodecane and redispersed before use.

In a flow cell the capsules were found to adhere to a streptavidin pretreated poly-styrene surface. Ultrasound pulses were delivered using a panametrix V302 transducer, 10 cycle burst, pulse repetition frequency 10 kHz (duty cycle 1%). In a typical field of view 200-400 air filled capsules accumulated. After exposure to ultrasound at a peak-peak setting of 400 mV no remaining capsules were seen, indicating acoustic activity of all adhering capsules.

### Example 4

### Partially Oil-Filled Capsules

Capsules were prepared from 0.25 g of a 5% solution of pla-pfo in dichloromethane, 0.5 g of 10% cyclodecane in dichloromethane and 0.5 g of 10% hexadecane in dichloromethane. The emulsion was washed 4 times to remove the excess pva. Centrifugation was used to separate the capsules from the liquid. In all washing steps the capsules were forming a foam layer indicating a lower density for the capsules than for water. 3% polyethylene glycol was added and the samples were lyophilized to remove the cyclodecane and redispersed before use.

Event count measurements showed a somewhat higher threshold than for capsules prepared without hexadecane, but the activation rate could not be distinguished from that of capsules prepared without hexadecane.

### Example 5

### Drug Loaded Contrast Agents

Pla-pfo was dissolved in dichloromethane to obtain a 5% (w/w) solution (solution A).

Paclitaxel, was dissolved in dichloromethane 10 mg/ml. 0.5 g of polymer solution, 1 g of the paclitaxel solution, 100 mg of hexadecane and 100 mg of cylodecane and 0.5 g of dichloromethane were mixed. This mixture was added to 10 g of 0.3% pva solution and emulsified by pressing the mixture through a glass filter. This was repeated 10 times after which the emulsion was stirred for one hour to evaporate the dichloromethane and complete capsule formation. The emulsion was washed 4 times to remove the excess pva. Centrifugation was used to separate the capsules from the liquid. In all washing steps the capsules were forming a foam layer indicating a lower density for the capsules than for water. 3% polyethylene glycol was added and the samples were lyophilized to remove the cyclodecane and redispersed before use.

The agent was redispersed in phosphate buffered saline (0.5 ml) giving a 10 mg paciltaxel/ml agent. Two 25 μl injections of the agent were performed for each mouse bearing two small MC38 (mouse colon adenocarcinoma) tumors symmetrically in the left and right hind leg regions.

A single element focused transducer was used for the delivery of therapeutic ultrasound (1 MHz, pulse length 300 μs and PRF 50 Hz). A custom-made gel cone with its tip pointing to a tumor was utilized as the acoustical coupling material between the transducer and the tumor.

A low-MI harmonic mode (opt:HGen with a tissue specific preset: vascular surgery/tumor) on a high-frequency probe CL15-7 connected to an ultrasound scanner (Philips HDI5000) was employed for monitoring the injection and 1 MHz ultrasound exposure. For imaging, a low MI of 0.15 was used for minimal bubble destruction. The imaging depth was 1.9 cm and the focus ˜1.5 cm.

After the first injection arrival of the agent in the tumor vasculature could be observed in the low-MI harmonic imaging mode, then therapeutic ultrasound was delivered for 10 seconds and consequently the desrupted agent could no longer be detected. After approximately 10 seconds inflow of fresh agent was again observed inside the tumor and then a destruction pulse was followed for roughly 10 seconds. In this way therapeutic ultrasound exposure was intermittently applied for 5 minutes. The above procedure was repeated after the second injection took place.

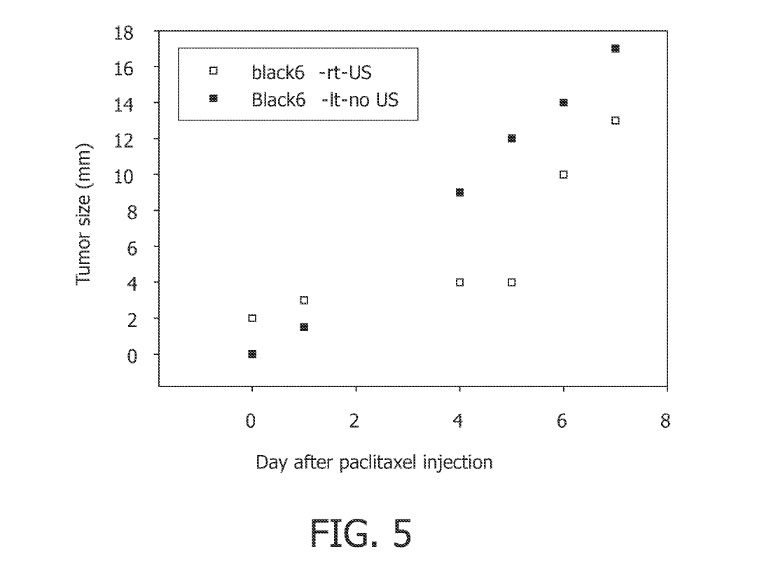

The tumor growth in the ultrasonically treated tumor was significantly delayed behind that of the untreated tumor as shown in FIG. 5 where the tumor size changes are shown with and without the application of therapeutic ultrasound.

1. Method for the production of particles comprising a gas core and a shell which method comprises the steps of:
a) providing a mixture comprising a shell composition, a first solvent (1) and a second non-solvent (2);
b) combining the mixture of step (a) with an aqueous composition thereby forming an emulsion of the mixture of step (a) in an aqueous phase;
c) applying conditions for volatizing solvent (1)
d) applying conditions for removal of water
e) applying conditions for removing of non-solvent (2)
characterized in that non-solvent (2) is selected from the group comprising organic compositions that have a vapor pressure significantly lower than water under the conditions of step (d).

2. Method according to claim 1 wherein non-solvent (2) has a vapor pressure which is at least 5 times lower than that of water.

3. Method according to claim 1 wherein non-solvent (2) is selected from the group comprising hydrocarbons comprising a carbon chain length of from 10 to 20 carbon atoms.

4. Method according to claim 1 wherein the non-solvent (2) is selected from the group comprising cyclodecane, decane, or a combination thereof.

5. Method according to claim 4 wherein the non-solvent (2) essentially consists of cyclodecane.

6. Method according to claim 1 wherein the shell composition is a polymer, preferably selected from the group comprising hydrophobic polymers.

7. Method according to claim 6 wherein the polymer composition comprises a polymer modified with at least one hydrophobic group that is preferably selected from the group comprising fluoride, alkyl chain comprising from 6 to 24 carbon atoms or a combination of these.

8. Method according to claim 7 wherein the polymer is selected from the group comprising polylactic acid with a perfluorinated moiety, polylactic-polyglycolacid copolymers, polycarpolactone, epsilon-caprolactone or a combination thereof.

9. Method according to claim 1 wherein in step (a) or (b) a stabilizing composition is included.

10. Method according to claim 1 wherein solvent (1) is a solvent having a vapor pressure higher than water under the conditions of step (c).

11. Method according to claim 1 wherein before step (c) the composition is supplemented with a composition comprising a therapeutic agent and/or a contrast agent, which agents are dissolved in at least one carrier liquid.

12. Method according to claim 11 wherein the carrier liquid comprises hexadecane.

13. Method according to claim 1, which comprises the inclusion of a therapeutic composition.

14. Method according to claim 1 wherein the particles have a shell with an average thickness of from 1 to 50 nm for an average radius of from 1 to 5 micrometer.

15. Particle comprising a gas core and a shell, which is obtained by the method according to claim 1.

16. Ultrasound diagnostic imaging contrast agent comprising at least a particle according to claim 15.

17. Therapeutic composition comprising at least one particle according to claim 15.

18. Ultrasound contrast agent according to claim 16, comprising a multitude of particles, wherein the majority of the particles, even more preferred from 80 to 100% of the particles are particles obtained by the method for the production of particles comprising a gas core and a shell which method comprises the steps of:
a) providing a mixture comprising a shell composition, a first solvent (1) and a second non-solvent (2);
b) combining the mixture of step (a) with an aqueous composition thereby forming an emulsion of the mixture of step (a) in an aqueous phase;
c) applying conditions for volatizing solvent (1)
d) applying conditions for removal of water
e) applying conditions for removing of non-solvent (2)
characterized in that non-solvent (2) is selected from the group comprising organic compositions that have a vapor pressure significantly louver than under the conditions of step (d).

19. Particle composition comprising a gas core and a polymeric shell wherein the particle has a diameter of from 0.1 to 5 micrometer and a shell thickness of from 1 to 80 nm, which particle can be acoustically activated by application of ultrasound at a mechanical index of at most 3.

20. Ultrasound contrast agent comprising polymer shelled particles according to claim 19, wherein at least 80%, preferably 80 to 100% of the particles are activated by ultrasound energy, in a mechanical index window of 0.5 units within the mechanical index range of 0.01 to 3.

21. Therapeutic composition comprising particles according to claim 19, which particles additionally comprise at least one drug composition.

22. Therapeutic composition according to claim 21 wherein at least 80%, preferably 80 to 100% of the particles are activated by ultrasound energy, in a mechanical index window of 0.5 units within the mechanical index range of 0.01 to 3.
